(19) United States
(12) Patent Application Publication
(10) Pub. No.: US 2026/0183511 A1
(43) Pub. Date: Jul. 2, 2026

(54) INTERLOCKING MECHANISM FOR EXTENDABLE URINARY CATHETER PRODUCTS

(71) Applicant: Hollister Incorporated, Libertyville, IL (US)

(72) Inventors: Luis Martins, Ballina (IE); Damien Biggins, Ballina (IE); Vincent Naughton, Sligo (IE); Jeffrey R. Burger, Bexley, OH (US); Chris W. Cicenas, Etna, OH (US)

(21) Appl. No.: 18/863,905

(22) PCT Filed: Apr. 28, 2023
(86) PCT No.: PCT/US2023/066383

§ 371 Date: Nov. 7, 2024

Related U.S. Application Data
(60) Provisional application No. 63/339,788, filed on May 9, 2022.

(51) Int. Cl. A61M 25/01

(52) U.S. Cl. CPC A61M 25/01; A61M 2025/0175; A61M 2202/0014; A61M 2202/0496; A61M 2210/1089

(57) ABSTRACT

Interlocking mechanisms (150) for extendable urinary catheters (20) having latched and keepers that engage to lock the catheter in the extended configuration.

The present application claims the benefit of and priority to U.S. Provisional Patent Application No. 63/339,788, filed May 9, 2022, which is hereby incorporated in its entirety herein by reference.

### DESCRIPTION

### Technical Field

The present disclosure generally relates to interlocking mechanisms for extendable urinary catheter products that lock the catheter in an extended, use configuration.

### Background

Catheters are used to treat many different types of medical conditions and typically include an elongated catheter tube inserted into and through a passageway or lumen of the body. Urinary catheters and, in particular, intermittent urinary catheters are commonly used by individuals who suffer from certain urinary system abnormalities, such as urinary incontinence. With the advent of intermittent urinary catheters, individuals with problems associated with the urinary system can conveniently self-catheterize to drain the individual's bladder.

Individuals who suffer from urinary incontinence will self-catheterize several times a day. Thus, they are required to carry intermittent urinary catheters with them wherever they go. Accordingly, it is desirable for urinary catheters to have compact and portable configurations that allow them to be discrete for storage and travel. The catheter also needs to have an extended configuration that is sufficient for catheterization.

There remains a need for improved catheter assemblies that move from a compact state to an extended configuration.

### SUMMARY

There are several aspects of the present subject matter which may be embodied separately or together in the devices and systems described and claimed below. These aspects may be employed alone or in combination with other aspects of the subject matter described herein, and the description of these aspects together is not intended to preclude the use of these aspects separately or the claiming of such aspects separately or in different combinations, as set forth in the claims appended hereto.

In one aspect, a urinary catheter has a collapsed configuration and an extended configuration. The urinary catheter includes a proximal tube having a proximal insertion end portion and a distal interlocking end portion. The proximal tube including at least one drainage opening in the proximal insertion end portion and a lumen extending from the at least one drainage opening to the distal interlocking end portion. The urinary catheter also includes a distal tube having a distal drainage end portion, a proximal interlocking end portion and a lumen extending from the proximal interlocking end portion to the distal drainage end portion. The proximal tube is displaceably arranged in the lumen of the distal tube such that the proximal tube and distal tube can be moved relative to each other from a collapsed configuration to an extended configuration. The catheter also includes an interlocking mechanism comprising a first interlocking member and a second interlocking member that engage to lock the proximal and distal tubes in the extended configuration. The first interlocking member being associated with the distal interlocking end portion of the proximal tube. The first interlocking member including a body having a proximal end, a distal end and a generally tubular shape having a longitudinal axis. There is at least one radially extending latch extending from the body of the first interlocking member. The second interlocking member is associated with the proximal interlocking end portion of the distal tube, the second interlocking member including at least one keeper. When the proximal and distal tubes are moved into the extended configuration, the at least one radially extending latch of the first interlocking mechanism and the keeper of the second interlocking mechanism engage to lock the proximal and distal tubes in the extended configuration and prevent the proximal and distal tubes from moving to the collapsed configuration.

### BRIEF DESCRIPTION OF FIGURES

FIG. 1 is a perspective view of an exemplary extendable urinary catheter shown in a collapsed, storage configuration;

FIG. 2 is a perspective view of the urinary catheter of FIG. 1, shown in the extended, use configuration;

FIG. 3 is a partial perspective, cross-sectional view of one embodiment of an extendable urinary catheter having an interlocking mechanism, shown with the catheter in the extended configuration and the interlocking mechanism in the locked state;

FIG. 4 is a perspective, exploded view of the interlocking mechanism of FIG. 3;

FIG. 5 is a cross-sectional view of another embodiment of an extendable catheter having an interlocking mechanism, shown with the catheter in the collapsed configuration and the interlocking mechanism in the unlocked state;

FIG. 6 is a cross-sectional view of the extendable catheter of FIG. 5, shown with the catheter in the extended configuration and the interlocking mechanism in the locked state;

FIG. 7 is a perspective, exploded view of the interlocking mechanism of FIG. 5;

FIG. 8 is a cross-sectional view of another embodiment of an extendable catheter having an interlocking mechanism, shown with the catheter in the collapsed configuration and the interlocking mechanism in the unlocked state;

FIG. 9 is a cross-sectional view of the extendable catheter of FIG. 8, shown with the catheter in the extended configuration and the interlocking mechanism in the locked state;

FIG. 10 is a perspective, exploded view of the interlocking mechanism of FIG. 8;

FIG. 11 is a cross-sectional view of another embodiment of an extendable catheter having an interlocking mechanism, shown with the catheter in the collapsed configuration and the interlocking mechanism in the unlocked state;

FIG. 12 is a cross-sectional view of the extendable catheter of FIG. 11, shown with the catheter in the extended configuration and the interlocking mechanism in the locked state;

FIG. 13 is a perspective, exploded view of the interlocking mechanism of FIG. 11;

FIG. 14 is a cross-sectional view of another embodiment of an extendable catheter having an interlocking mechanism, shown with the catheter in the collapsed configuration and the interlocking mechanism in the unlocked state;

FIG. 15 is a cross-sectional view of the extendable catheter of FIG. 14, shown with the catheter in the extended configuration and the interlocking mechanism in the locked state;

FIG. 16 is a perspective, exploded view of one of the components of the interlocking mechanism of FIG. 14;

FIG. 17 is a partial perspective view of the catheter of FIG. 14;

FIG. 18 is a partial perspective view of the catheter of FIG. 14;

FIG. 19 is a cross-sectional view of another embodiment of an extendable catheter having an interlocking mechanism, shown with the catheter in the collapsed configuration and the interlocking mechanism in the unlocked state;

FIG. 20 is a cross-sectional view of the extendable catheter of FIG. 19, shown with the catheter in the extended configuration and the interlocking mechanism in the locked state;

FIG. 21 is a partial perspective view of the catheter of FIG. 19;

FIG. 22 is a partial cross-sectional view of another embodiment of an extendable catheter having an interlocking mechanism, shown with the catheter in the collapsed configuration and the interlocking mechanism in the unlocked state;

FIG. 23 is a side perspective view of the interlocking member shown FIG. 22;

FIG. 24 is a rear perspective view of the interlocking member of FIG. 22;

FIG. 25 is a front perspective view of the interlocking member of FIG. 22; and

FIGS. 26-30 are cross-sectional views illustrating the locking mechanism moving from the initial position to the locked position.

### DETAILED DESCRIPTION OF THE ILLUSTRATED EMBODIMENTS

The embodiments disclosed herein are for the purpose of providing a description of the present subject matter, and it is understood that the subject matter may be embodied in various other forms and combinations not shown in detail. Therefore, specific embodiments and features disclosed herein are not to be interpreted as limiting the subject matter defined in the accompanying claims.

The present disclosure is directed to urinary catheter products wherein the catheter has a collapsed, storage configuration and an extended, use configuration.

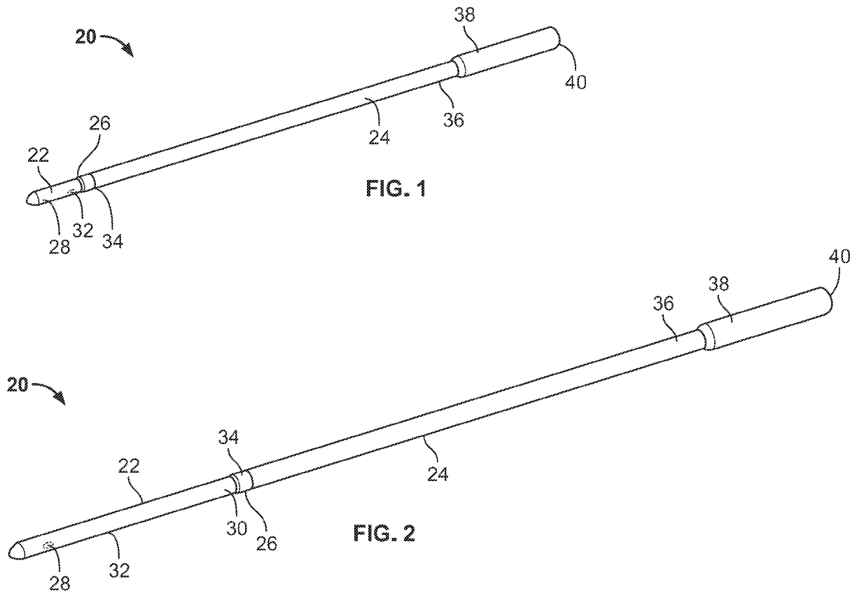

FIGS. 1 and 2 illustrate one exemplary embodiment of an extendable urinary catheter 20 for use in the catheter products of the present disclosure. The catheter 20 may be a telescoping catheter that includes a proximal tube 22 and distal tube 24. The proximal and distal tubes 22 and 24 may be in telescopic relation to one another and/or connected to each other by a telescopic joint 26. In the illustrated embodiment, the proximal tube 22 and the distal tube 24 are concentric and slide relative to one another. The proximal tube 22 is located and slidable within a lumen of the distal tube 24. In other alternatives, the distal tube 24 could be located and slidable within a lumen of the proximal tube 22.

FIG. 1 shows catheter 20 in a collapsed, storage configuration. In the collapsed configuration, a majority of the proximal tube 22 and a majority of the distal tube 24 overlap one another. In the illustrated embodiment, the majority of proximal tube 22 is located within a lumen of the distal tube 24. Catheter 20 can be moved from the collapsed configuration shown in FIG. 1 to the extended configuration shown in FIG. 2. To move catheter 20 into the extended configuration, the proximal tube 22 and distal tube 24 are slid relative to one another in an extending telescoping fashion. As shown in FIG. 2, the axial length of the catheter 20 in the extended configuration is larger than the axial length of the catheter 20 in the collapsed configuration. When in the extended configuration, catheter 20 is ready for use. In use, the telescopic joint 26 joining the proximal and distal tubes 22 and 24 may or may not be configured to be insertable into the urethra.

The proximal tube 22 of the catheter 20 includes a proximal insertion end portion 28 and a distal interlocking end portion 30 (FIG. 2). The proximal insertion end portion 28 includes at least one drainage openings or eyelets 32 for draining urine from the bladder. The eyelets 32 communicate with a drainage lumen (not shown) of the proximal tube 22. The drainage lumen of the proximal tube 22 communicates with the lumen (not shown) of the distal tube 24. The lumens of the proximal tube 22 and the distal tube 24 define the drainage lumen of the catheter 20.

The distal tube 24 includes a proximal interlocking end portion 34 and a distal drainage end portion 36. A drainage member 38 may be located at the distal end drainage portion 36 of the distal tube 24. The drainage member 38 may be a funnel or connector with a drainage outlet 40 for directing urine drainage out of the catheter 20.

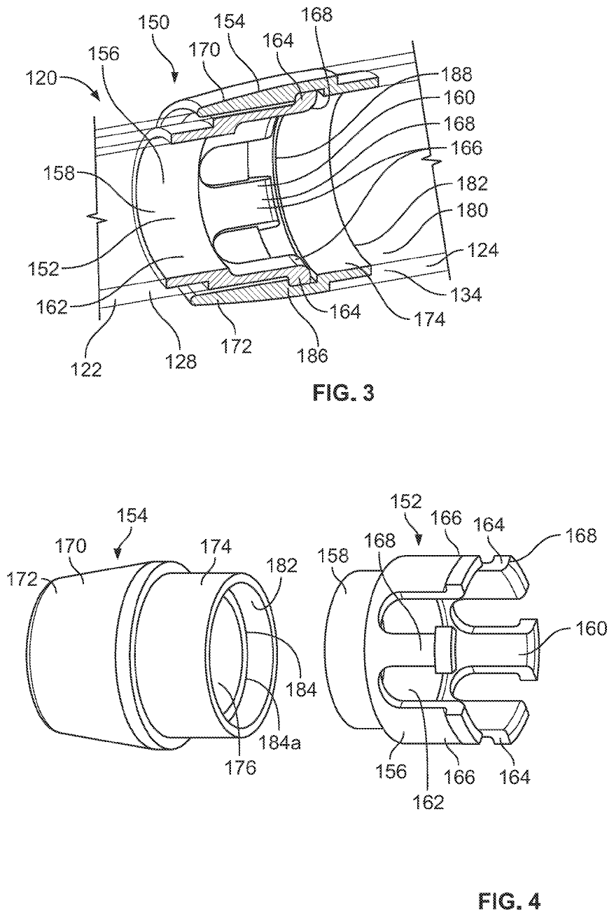

FIGS. 3 and 4 illustrate an extendable catheter 120 having an interlocking mechanism 150. Similar to FIGS. 1 and 2, catheter 120 has a proximal tube 122 and a distal tube 124. FIG. 3 shows the catheter 120 in the extendable state and the interlocking mechanism 150 in a locked state. In the locked state, the interlocking mechanism 150 has sufficient strength to prevent the catheter 120 from returning to the collapsed configuration during use. The interlocking mechanism 150 also prevents the proximal tube 122 and distal tube 124 from separating.

The interlocking mechanism 150 includes a first interlocking member 152 and a second interlocking member 154, which engage each other to lock the catheter 120 in an extended position. The first interlocking member 152 is associated with the distal interlocking end portion 128 of the proximal tube 122. In the illustrated embodiment, the first interlocking member 152 has a body 156 that includes a proximal end 158 and a distal end 160. The body 156 has a generally tubular shape with a longitudinal axis and a lumen 162 therethrough. The proximal end 158 of the first interlocking member 152 is connected to the distal interlocking end portion 128 of proximal tube 122. In one alternative, the distal interlocking end portion 128 of the proximal tube 122 is over-molded over the proximal end 158 of the first interlocking member 152. In another option, the proximal end 158 of the first interlocking member 152 is embedded in the distal interlocking end portion 128 of the proximal tube 122. In yet another alternative, the distal interlocking end portion 128 of the proximal tube 122 is friction or heat fit over the proximal end 158 of the first interlocking member 152.

The first interlocking member 152 includes one or more latches 164 extending radially outward from the body 156. In the illustrated embodiment, body 156 includes a plurality of distally extending arms 166, which have the latches thereon. The latches 164 may be located at the distal end 168 of the arms 166 or any other location on arms 166. The arms 166 may be elastic such that they may be radially compressed and then return to their natural resting state.

The second interlocking member 154 includes a generally tubular body 170 with a proximal end portion 172, a distal end 174, and a lumen 176. The distal end 174 of the body 170 of the second interlocking member 154 is connected to the proximal interlocking end portion 134 of the distal tube 124. In one alternative, the proximal interlocking end portion 134 of the distal tube 124 is overmolded over the distal end 174 of the second interlocking member 154. In another alternative, the distal end 174 of the second interlocking member 154 is embedded in the proximal interlocking end portion 134 of the distal tube 124. In yet another alternative, the proximal interlocking end portion 134 of the distal tube 124 is friction or heat fit over the distal end 174 of the second interlocking member 154. In the illustrated embodiment, the distal end 174 of the second interlocking member 154 and the proximal interlocking end portion 134 of the distal tube 124 have a joint that may be a lap-joint or has a generally L-shaped cross-section. This joint provides a smooth transition between the inner surface 180 of the distal tube 124 and the inner surface 182 of the second interlocking member 154.

The body 170 of the second interlocking member 154 may vary in outer diameter. For example, the distal end 174 may have smaller outer diameter than the proximal end portion 172. Furthermore, the proximal end portion 172 may be tapered radially inwardly in a direction toward the proximal end of the body 170. This inward taper may result in a smooth and/or gradual transition between the outer surface of the proximal tube 122 and the outer surface of the body 170 of the second interlocking member 154. The inner surface 182 of the second interlocking member 154 may have a keeper 184 that engages the one or more latches 164 of the first interlocking member 152 to lock the catheter 120 in the extended configuration. In the illustrate embodiment, one or more internal depression 184a in the in inner surface 182 of the second interlocking member 154 defines the keeper. For example, the internal depression 184a may be a channel or groove in the inner surface 182. The channel may continuous. In other alternatives, internal depressions 184a may be a plurality of depressions in the inner surface 182.

The extendable catheter 120 is stored in the collapsed configuration. For example, the catheter 120 may be distributed to the user in the stored configuration contained in a package. After the user opens the package, the user moves the proximal and distal tubes 122, 124 away from each other, moving the proximal tube 122 out of the lumen of the distal tube 124. The inner diameter of the distal tube 124 is smaller than the outer diameter of the distal end 160 of the first interlocking member 152. Because of this arrangement, the distal end 160 of the first interlocking member 152 is compress radially inwardly. In the illustrated embodiment, latches 164 of the elastic arms 166, extending radially outwardly, are in contact with the inner surface 180 of the distal tube 124. This results in the elastic arms 166 being compress radially inwardly. The proximal and distal tubes 122, 124 are moved away from each other until the latches 164 engage the keeper 184. In the illustrated embodiment, when the latches 164 reach the keeper 184, the arms 166 move outwardly toward their natural, uncompressed state. The latches 164 move into the channel, engaging the keeper 184. The proximal shoulder 186 of the channel defines a stop or stop mechanism, wherein engagement between the latch(es) 164 and shoulder 186 prevents removal/separation of the proximal tube 122 from the distal tube 124. Engagement between the latch(es) 164 and the distal shoulder 188 prevents the urinary catheter 120 from collapsing. The user then preforms catheterization with the catheter 120 by inserting the catheter into the urethra. During catheterization, the distal tube 124 may have an insertable portion that is inserted into the urethra.

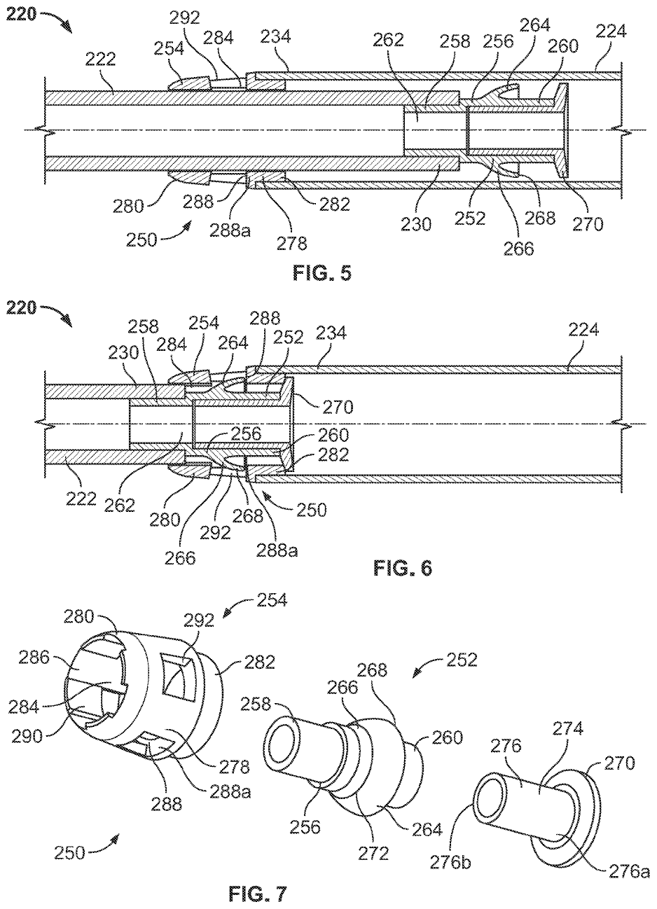

FIGS. 5-7 illustrate another alternative extendable urinary catheter 220 having an alternative interlocking mechanism 250. Similar to FIGS. 1 and 2, the catheter 220 has a proximal tube 222 and a distal tube 224. FIG. 5 shows the catheter 220 in a collapsed configuration and FIG. 6 shows the catheter 220 in the extended configuration and the interlocking mechanism 250 in a locked state. In the locked state, the interlocking mechanism 250 has sufficient strength to prevent the catheter 220 from returning to the collapsed configuration during use. The interlocking mechanism 250 also prevents the proximal tube 222 and distal tube 224 from separating.

The interlocking mechanism 250 includes a first interlocking member 252 and a second interlocking member 254, which engage each other to lock the catheter 220 in an extended position. The first interlocking member 252 is associated with the distal interlocking end portion 230 of the proximal tube 222. In the illustrated embodiment, the first interlocking member 252 has a body 256 that includes a proximal end 258 and a distal end 260. The body 256 has a generally tubular shape, having a longitudinal axis and a lumen 262 therethrough. The proximal end 258 of the first interlocking member 252 is connected to the distal interlocking end portion 230 of proximal tube 222. Similar to that of FIG. 3, the proximal end 258 of the interlocking member 252 and distal interlocking end portion 230 of proximal tube 222 may be connected by over-molding, embedding, friction fit, heat fit, etc.

The first interlocking member 252 includes one or more latches 264 extending radially outward from the body 256. In the illustrated embodiment, the latch 264 has a continuous wall with a generally truncated conical shape. The proximal end 266 of the latch 264 extends from the body 256. The latch 264 flares radially outwardly to a distal end 268 having a larger diameter than the proximal end 266. The latch 264 may be elastic such that it may be radially compressed and then return to its natural, uncompressed state.

The body 256 of the first interlocking member 252 also includes a flange 270 that defines a stop or stop mechanism. In the illustrated embodiment, flange 270 is located at the distal end 260 of the body 256 of the first interlocking member 252. The first interlocking member 252 may be a unitary piece, such that the flange 270 is integral with the remainder of the body 256. In another alternative and as shown in the figures, the body 256 of the first interlocking member 252 may be made from two separate and distinct components. For example, the body 256 may include a proximal component 272 and a distal component 274, wherein the flange 270 is part of the distal component 274. In the illustrated embodiment, the distal component 274 has a tubular post 276 and the flange 270 is located at the distal end 276a of the tubular post 276. The proximal end 276b of the tubular post 276 is inserted into the lumen of the proximal component 272 and attached thereto, by for example friction fit, heat fit, adhesive, etc.

The second interlocking member 254 includes a generally tubular body 278 having a proximal end 280, a distal end 282 and a lumen 284. The distal end 282 of the body 278 of the second interlocking member 254 is connected to the proximal interlocking end 234 of the distal tube 224. Similar to that of FIG. 3, the distal end 282 of the interlocking member 254 and proximal interlocking end 234 of distal tube 224 may be connected by over-molding, embedding, friction fit, heat fit, etc.

The body 278 of the second interlocking member 254 may vary in outer diameter. For example, the distal end portion 282 may have smaller outer diameter than the proximal end portion 280. Furthermore, the proximal end portion 280 may be taper radially inwardly in a direction toward the proximal end of the body 278. This inward taper may result in a smooth and/or gradual transition between the outer surface of the proximal tube 222 and the outer surface of the body 278 of the second interlocking member 254.

The inner surface 286 of the second interlocking member 254 may have a keeper 288 that engages the one or more latches 264 of the first interlocking member 252 to lock the catheter 220 in the extended configuration. In the illustrate embodiment, the inner surface 286 of the second interlocking member 254 may include one or more shoulders 288a, which may be steps or ledges, that define the keeper 288. For example, the inner diameter of the second interlocking member 254 may vary in diameter. In the illustrated embodiment, an inner diameter of a distal end portion 282 of the second interlocking member 254 may be less than an inner diameter of a proximal end portion 280. The transition between these portions is stepped to create a shoulder 288a or surface to which the longitudinal axis of the second interlocking member 254 is normal. In other embodiments, the keeper 288 may be defined by a plurality of projections extending from the inner surface 286 and projecting into the lumen of the second interlocking member 254.

Referring to FIG. 7, the inner surface 286 of the second interlocking member 254 may also include a spacers or shims 290 that extend from the inner surface 286 into the lumen. In the illustrated embodiment, the spacers 290 are uniformly space about the circumference of the lumen of the second interlocking member 254. The spacers 290 contact, center and steady the proximal tube 222 as it passes through the second interlocking member 254. Referring to FIGS. 6 and 7, the body 278 of the second interlocking member 254 may also have a window or opening 292 that allows the user to visualize whether the interlocking members are engaged.

The extendable catheter 220 is stored in the collapsed configuration. For example, the catheter 220 may be distributed to the user packaged in the collapsed configuration. After the user opens the package, the user moves the proximal and distal tubes 222, 224 away from each other, moving the proximal tube out 222 of the lumen of the distal tube 224. The maximum outer diameter of the cone shaped latch 264 is larger than the inner diameter of the distal end 282 of the second interlocking member 254. As the user continues to move the proximal and distal tubes 222,224 apart, the cone-shaped latch 264 of the first interlocking member 252 contacts the inner surface 286 of the second interlocking member 254. The outer surface of the latch 264 acts as a ramp as it slides against the inner surface 286 of the second interlocking member 254. As the larger diameter distal end portion 268 of the latch 264 enters the lumen 284 of the second interlocking member 254, the latch 264 is compressed radially inwardly so as to pass through the lumen 284. When the distal end 268 of the latch 264 moves passed the keeper 288, which in this embodiment is a shoulder 288a, the latch 264 expends back towards its normal, uncompressed state. The distal end 268 of the latch 264 engages the shoulder 288a of the keeper 288 to lock the catheter 220 in the extended configuration. Furthermore, the flange 270 has a larger diameter than the inner diameter of the distal end 282 of the second interlocking member 254. The flange 270 and distal end 282 of the second interlocking member 254 define a stop or stop mechanism that prevents the tubes 222, 224 from separating. The distance between the latch 164 and the flange 270 are such that there is minimal relative movement between the two tubes 222, 224. The user then preforms catheterization with the catheter 220 by inserting the catheter into the urethra. During catheterization, the distal tube 224 may have an insertable portion that is inserted into the urethra.

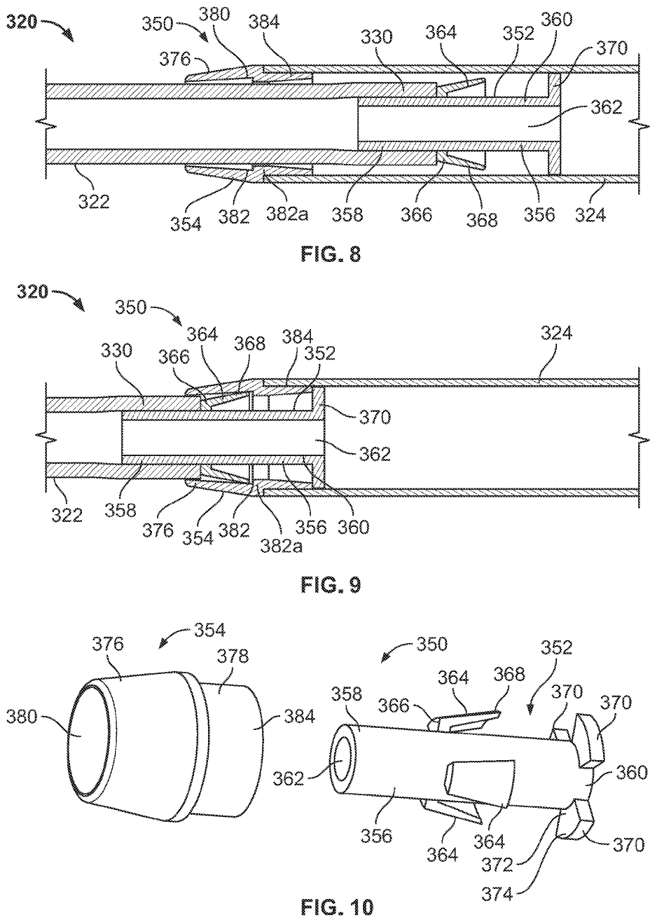

FIGS. 8-10 illustrate another alternative extendable urinary catheter 320 having an alternative interlocking mechanism 350. Similar to FIGS. 1 and 2, the catheter 320 has a proximal tube 322 and a distal tube 324. FIG. 8 shows the catheter 320 in a collapsed configuration and FIG. 9 shows the catheter 320 in the extended configuration and the interlocking mechanism 350 in a locked state. In the locked state, the interlocking mechanism 350 has sufficient strength to prevent the catheter 320 from returning to the collapsed configuration during use. The interlocking mechanism 350 also prevents the proximal tube 322 and distal tube 324 from separating.

The interlocking mechanism 350 includes a first interlocking member 352 and a second interlocking member 354, which engage each other to lock the catheter 220 in an extended position. The first interlocking member 352 is associated with the distal interlocking end portion 330 of the proximal tube 322. In the illustrated embodiment, the first interlocking member 352 has a body 356 that includes a proximal end 358 and a distal end 360. The body 356 has a generally tubular shape, having a longitudinal axis and a lumen 362 therethrough. The proximal end 358 of the first interlocking member 352 is connected to the distal interlocking end portion 330 of proximal tube 322. Similar to that of FIG. 3, the proximal end 358 of the interlocking member 352 and distal interlocking end portion 330 of proximal tube 322 may be connected by over-molding, embedding, friction fit, heat fit, etc.

The first interlocking member 352 is similar to the interlocking member 252 of FIGS. 5-8, except that it has a plurality of latches 364 and a plurality of radial distal projections 370. The illustrated embodiment includes three discrete latches 364 uniformly spaced about the body 356. However, in other alternatives, there could be more or less than three latches 364. The latches 364 have a proximal end 366 extending from the body 356 and flare radially outwardly toward the distal end 368. The body 356 also includes three discrete radial distal projections 370. However, in other alternatives, there could be more or less than three radial distal projections 370. The projections 370 extend radially outwardly from the body 356. The projections 370 have one end 372 extending from the body 356 and another free end 374 that is located radially outward from the body 356.

The second interlocking member 354 is similar to the interlocking member 254 of FIGS. 5-8, except that it does not have any windows or opening. Optionally, the interlocking member 354 could include such windows. Also optionally, the inner surface of the interlocking member 354 could include the above-mentions spacers/shims. Furthermore, the outer surface proximal end portion 376 of the body 378 of the second interlocking member 354 may be taper radially inwardly in a direction toward the proximal end of the body 378. This inward taper may result in a smooth and/or gradual transition between the outer surface of the proximal tube 322 and the outer surface of the body 378 of the second interlocking member 354.

The inner surface 380 of the second interlocking member 354 may have a keeper 382 that engages the latches 364 of the first interlocking member 352 to lock the catheter in the extended configuration. In the illustrate embodiment, the inner surface 380 of the second interlocking member 354 may include one or more shoulders 382a, which may be steps or ledges, that define the keeper 382. For example, the inner diameter of the second interlocking member 354 may vary. In the illustrated embodiment, an inner diameter of a distal end 384 of the second interlocking member may be less than an inner diameter of a proximal portion 376. The transition between these portions is stepped to create a shoulder or surface 382a to which the longitudinal axis of the second interlocking member 354 is normal. In other embodiments, the keeper 382 may be defined by a plurality of projections extending from the inner surface 380 and projecting into the lumen.

The extendable catheter 320 may be packaged and stored in the collapsed configuration. After the user opens the package, the user moves the proximal and distal tubes 322, 324 away from each other, moving the proximal tube 322 out of the lumen of the distal tube 324. The maximum combined outer radial outer boundary defined by the latches 364 is larger than the inner diameter of the distal end 384 of the second interlocking member 354. As the user continues to move the proximal and distal tubes 322, 324 apart, the latches 364 of the first interlocking member 352 contact the inner surface 380 of the second interlocking member 354. The outer surfaces of the latches 364 act as ramps as they slide against the inner surface 380 of the second interlocking member 354. As the latches 364 enter the lumen of the second interlocking member 354, the latches 364 compress or bend radially inwardly so as to pass through the lumen. When the distal ends 368 of the latches 364 move past the keeper 382, which in this embodiment is a shoulder 382a, the latches 364 expend back towards their normal, uncompressed state. The distal ends 368 of the latches 364 engage the shoulder 382a of the keeper 382 to lock the catheter 320 in the extended configuration. Furthermore, the radial projections 370 have a larger radial boundary than the inner diameter of the distal end 384 of the second interlocking member 354. The projections 370 and distal end 384 of the second interlocking member 354 contact each other to prevent the tubes 322, 324 from separating. The distance between the latches 364 and the projections 370 are such that there is minimal relative movement between the two tubes 322, 324. The user then preforms catheterization with the catheter 320 by inserting the catheter 320 into the urethra. During catheterization, the distal tube 324 may have an insertable portion that is inserted into the urethra.

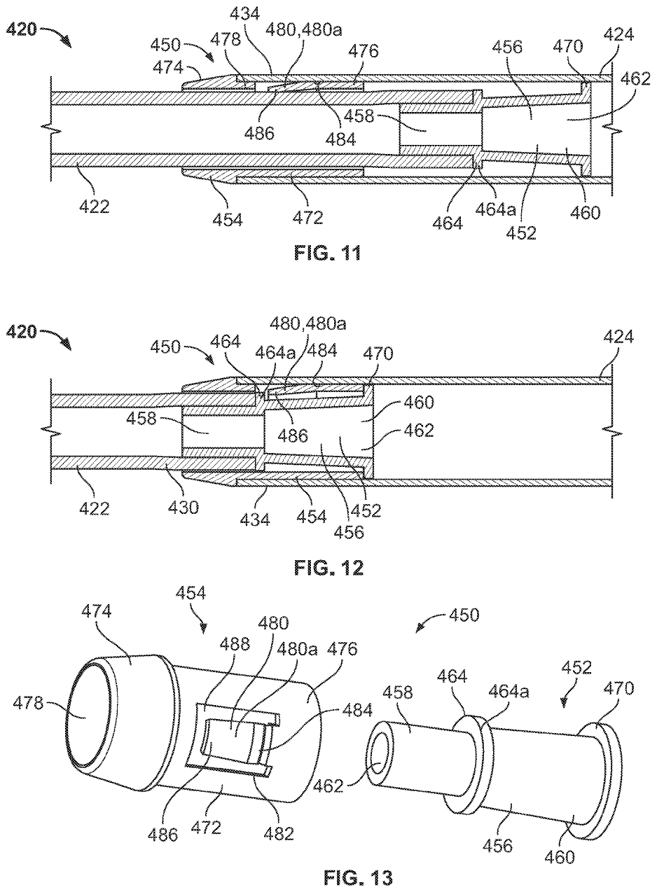

FIGS. 11-13 illustrate another alternative extendable urinary catheter 420 having an alternative interlocking mechanism 450. Similar to FIGS. 1 and 2, the catheter 420 has a proximal tube 422 and a distal tube 424. FIG. 11 shows the catheter 420 in a collapsed configuration and FIG. 12 shows the catheter 420 in the extended configuration and the interlocking mechanism 450 in a locked state. In the locked state, the interlocking mechanism 450 has sufficient strength to prevent the catheter 420 from returning to the collapsed configuration during use. The interlocking mechanism 450 also prevents the proximal tube 422 and distal tube 424 from separating.

The interlocking mechanism 450 includes a first interlocking member 452 and a second interlocking member 454, which engage each other to lock the catheter 420 in an extended position. The first interlocking member 452 is associated with the distal interlocking end portion 430 of the proximal tube 422. In the illustrated embodiment, the first interlocking member 452 has a body 456 that includes a proximal end 458 and a distal end 460. The body 456 has a generally tubular shape, having a longitudinal axis and a lumen 462 therethrough. The proximal end 458 of the first interlocking member 452 is connected to the distal interlocking end portion 430 of proximal tube 422. Similar to that of FIG. 3, the proximal end 458 of the interlocking member 452 and distal interlocking end portion 430 of proximal tube 422 by be connected by over-molding, embedding, friction fit, heat fit, etc.

The first interlocking member 452 includes a latch 464 extending radially outward from the body 456. In the illustrated embodiment, the latch 464 is a flange or lip 464a extending radially outwardly from the body 456.

The body 456 of the first interlocking member 452 also includes a second distal flange 470. In the illustrated embodiment, distal flange 470 is located at the distal end 460 of the first interlocking member 452. The first interlocking member 452 may be a unitary piece, such that the flange 470 is integral with the remainder of the body 456. In another alternative, the body 456 of the first interlocking member 452 may be made from two separate and distinct components, like that shown in FIG. 7. For example, the body 456 may include a proximal component and a distal component, wherein the flange 470 is part of the distal component. The distal component may have a tubular post and the flange 470 at the distal end of the tubular post. The proximal end of the tubular post is inserted into the lumen of the proximal component and attached thereto, by for example friction fit, heat fit, adhesive, etc.

The second interlocking member 454 includes a generally tubular body 472 having a proximal end portion 474, a distal end 476 and a lumen 478 passing therethrough. The distal end 476 of the body 456 of the second interlocking member 454 is inserted into the lumen of the proximal interlocking end portion 434 of the distal tube 424 and the second interlocking member 454 is attached thereto. Similar to that of FIG. 3, the distal end 476 of the second interlocking member 454 and proximal interlocking end portion 434 of distal tube 424 may be connected by over-molding, embedding, friction fit, heat fit, etc.

The body 472 of the second interlocking member 454 may vary in outer diameter. For example, the distal end portion 476 may have smaller outer diameter than the proximal end portion 474. Furthermore, the proximal end portion 474 may be taper radially inwardly in a direction toward the proximal end of the body 472. This inward taper may result in a smooth and/or gradual transition between the outer surface of the proximal tube 424 and the outer surface of the body 472 of the second interlocking member 454.

The second interlocking member 454 may have a keeper 480 that engages the latch 464 of the first interlocking member 452 to lock the catheter 420 in the extended configuration. In the illustrate embodiment, the keeper 480 of the second interlocking member 454 may include one or more flexible arms 480a that extend at an angle from the wall 482 of the second interlocking member 454 through a window or opening 488 into the lumen 478. The flexible arm 480 a has a distal portion 484 extending from the wall 482 and a proximal free end 486 extending into the lumen 478. Optionally, the inner surface may also include a spacers or shims that extend from the inner surface into the lumen, like those shown in FIG. 7.

The extendable catheter 420 may be stored and packaged in the collapsed configuration. After the user opens the package, the user moves the proximal and distal tubes 422, 424 away from each other, moving the proximal tube 422 out of the lumen of the distal tube 424. The outer diameter of latch's flange 464, 464a is larger than the inner diameter of the lumen 478 of the second interlocking member 454 in at least the segment of the body 472 having the flexible arm 480a defining the keeper 480. As the user continues to move the proximal and distal tubes 422, 424 apart, the flange 464a of the first interlocking member 452 contacts the flexible arm 480a defining the keeper 480 of the second interlocking member 454. As the flange 464a defining the latch 464 enters the lumen 478 of the second interlocking member 454, the flange 464a is pulled with sufficient force to move the flexible arm 480a outward, allowing the flange 464a to pass. When the flange 464a has moved passed proximal end 486 of the flexible arm 480a of the keeper 480, the arm 480a moves back towards its original position. The distal surface of the flange 464a engages the proximal end 486 of the arm 480a to lock the catheter 420 in the extended configuration. Furthermore, the second flange 470 has a larger outer diameter than the inner diameter of the distal end 476 of the second interlocking member 454. The second flange 470 and distal end 476 of the second interlocking member 454 define a stop or stop mechanism that prevents the tubes 422, 424 from separating. The tolerances between the latch 464 and the second flange 470 are such that there is minimal relative movement between the two tubes 422, 424. The user then preforms catheterization with the catheter 420 by inserting the catheter into the urethra. During catheterization, the distal tube 424 may have an insertable portion that may be inserted into the urethra.

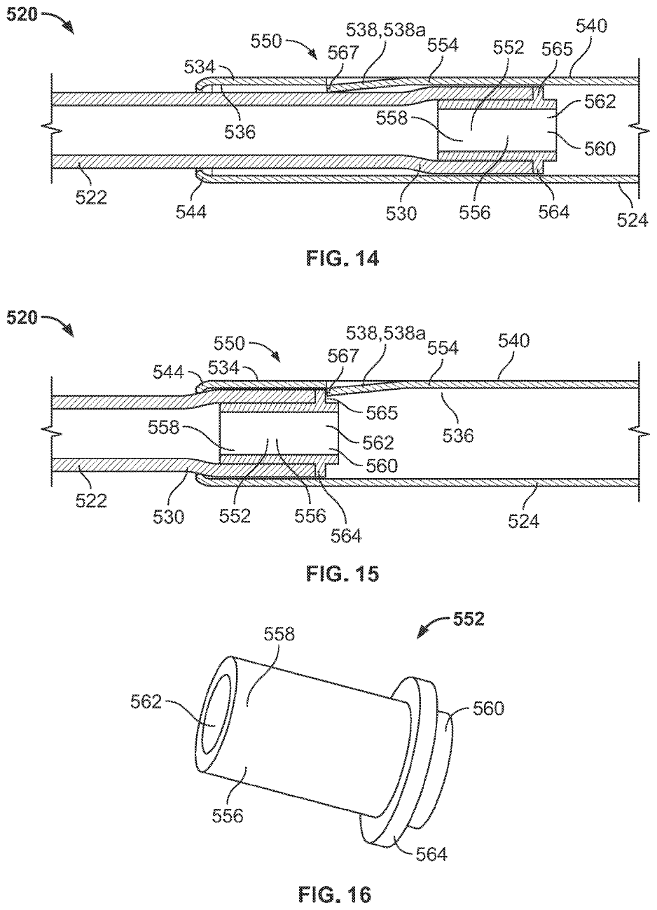

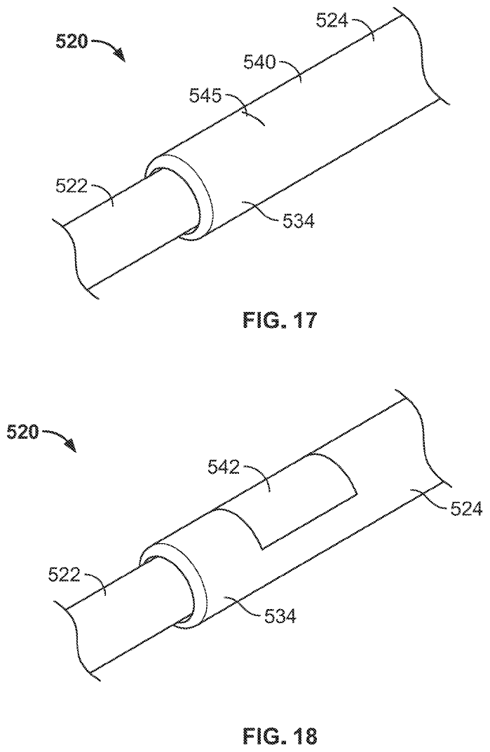

FIGS. 14-18 illustrate another alternative extendable urinary catheter 520 having an alternative interlocking mechanism 550. Similar to FIGS. 1 and 2, the catheter 520 has a proximal tube 522 and a distal tube 524. FIG. 14 shows the catheter 520 in a collapsed configuration and FIG. 15 shows the catheter 520 in the extended configuration and the interlocking mechanism 550 in a locked state. In the locked state, the interlocking mechanism 550 has sufficient strength to prevent the catheter 520 from returning to the collapsed configuration during use. The interlocking mechanism 550 also prevents the proximal tube 522 and distal tube 524 from separating.

The interlocking mechanism 550 includes a first interlocking member 552 and a second interlocking member 554, which engage each other to lock the catheter 520 in an extended position. The first interlocking member 552 is associated with the distal interlocking end portion 530 of the proximal tube 522. In the illustrated embodiment, the first interlocking member 552 has a body 556 that includes a proximal end 558 and a distal end 560. The body 556 has a generally tubular shape with a longitudinal axis and a lumen 562 therethrough. The proximal end 558 of the first interlocking member 552 is connected to the distal interlocking end portion 530 of proximal tube 522. Similar to that of FIG. 3, the proximal end 558 of the interlocking member 552 and distal interlocking end portion 530 of proximal tube 522 may be connected by over-molding, embedding, friction fit, heat fit, etc.

The first interlocking member 552 includes a latch 564 extending radially outward from the body 556. In the illustrated embodiment, the latch 564 is a flange or lip extending radially outwardly from the body 556. Although the latch 564 is shown near the distal end 560 of the body 556, the latch 564 may be located anywhere along the body.

The second interlocking member 554 is defined by the proximal interlocking end portion 534 of the distal tube 524. The inner surface 536 of the proximal end portion 534 includes projection 538a extending into the lumen, wherein the projection 538a defines the keeper 538. The projection 538a may be integral with the wall of the tubing 524. In one alternative, the projection 538a may be formed by deformation of the tube wall. In one embodiment, the wall may be depressed from the outer surface 540, thereby forming the projection 538a defining the keeper 538 on the inner surface 536 of the tube wall. As shown in FIG. 18, when the outer wall of the tube 524 is depressed or deformed, the distal tube 524 may include a cap or cover 542 to cover the depression 545. The cap 542 may be attached in any suitable manner, such as adhesive, snap fit, etc.

The inner diameter of the proximal interlocking end portion 534 of the distal tube 524 may be smaller than the outer diameter of the proximal end portion 558 of the body 556 of the first interlocking member 552. For example, the proximal terminal end 544 of the distal tube 524 may be flared inwardly.

The extendable catheter 520 is stored and packaged in the collapsed configuration, as shown in FIG. 14. After the user opens the package, the user moves the proximal and distal tubes 522, 524 away from each other, moving the proximal tube 522 out of the lumen of the distal tube 524. The diameter of latch's flange 564 is larger than the inner diameter of the lumen of the distal tube 524 in at least the segment of tube 524 containing the projection 538a defining the keeper 538. As the user continues to move the proximal and distal tubes 522, 524 apart, the flange defining the latch 564 of the first interlocking member 552 contacts the projection 538a defining the keeper 538 of the second interlocking member 554. The tubes 522, 524 are pulled apart with sufficient force to deform the latch 564, deform the projection 538a or both deform the latch and the projection, allowing the latch 564 to pass the projection 538a. When the latch 564 has moved passed proximal end of the projection 538a, the projection 538a and/or the latch 564 move back towards their original configurations. The distal surface 565 of the latch 564 engages the proximal end 567 of the projection 538a to lock the catheter 520 in the extended configuration. Furthermore, the proximal end 558 of the body 556 of the first interlocking member 552 contacts the proximal end 534 of the distal tube 524, which acts as a stop or stop mechanism to prevent the tubes 522, 524 from separating. The distance between the latch and the stop is such that there is minimal relative movement between the two tubes. The user then preforms catheterization with the catheter by inserting the catheter into the urethra. During catheterization, the distal tube 524 may have an insertable portion that may be inserted into the urethra.

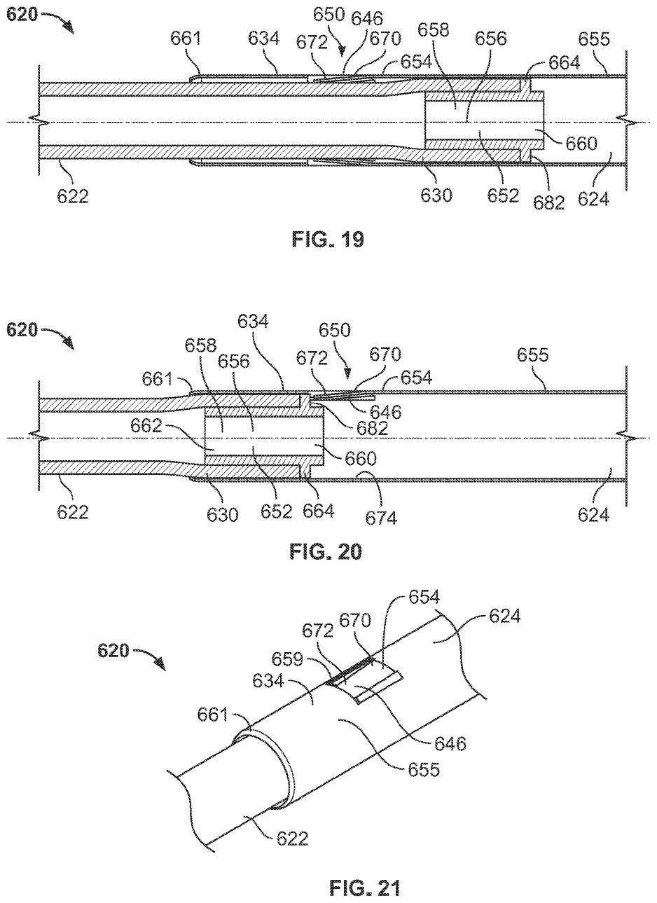

FIGS. 19-21 illustrate another alternative extendable urinary catheter 620 having an alternative interlocking mechanism 650. Similar to FIGS. 1 and 2, the catheter 620 has a proximal tube 622 and a distal tube 624. FIG. 19 shows the catheter 620 in a collapsed configuration and FIG. 20 shows the catheter 620 in the extended configuration and the interlocking mechanism 650 in a locked state. In the locked state, the interlocking mechanism 650 has sufficient strength to prevent the catheter 620 from returning to the collapsed configuration during use. The interlocking mechanism 650 also prevents the proximal tube 622 and distal tube 624 from separating.

The interlocking mechanism 650 includes a first interlocking member 652 and a second interlocking member 654, which engage each other to lock the catheter 620 in an extended position. The first interlocking member 652 is like that shown in FIG. 16 and is associated with the distal interlocking end portion 630 of the proximal tube 622. In the illustrated embodiment, the first interlocking member 652 has a body 656 that includes a proximal end 658 and a distal end 660. The body 656 has a generally tubular shape, having a longitudinal axis and a lumen 662 therethrough. The proximal end 658 of the first interlocking member 652 is connected to the distal interlocking end portion 630 of proximal tube 622. Similar to that of FIG. 3, the proximal end 658 of the interlocking member 652 and distal interlocking end portion 630 of proximal tube 622 may be connected by over-molding, embedding, friction fit, heat fit, etc.

The first interlocking member 652 includes a latch 664 extending radially outward from the body 656. In the illustrated embodiment, the latch 664 is a flange or lip extending radially outwardly from the body. Although the latch 664 is shown near the distal end 660 of the body 656, the latch 664 may be located anywhere along the body.

The second interlocking member 654 is defined by the proximal interlocking end portion 634 of the distal tube 624. The tube wall 655 of the distal end portion 634 includes a keeper 646. The keeper 646 is a projection extending into the lumen of the distal tube 624. The projection defining the keeper 646 may be a flexible arm embedded or otherwise attached to the wall 655 of the tubing 624. The flexible arm may be metal or plastic. Referring to FIG. 21, the proximal interlocking end 634 of the distal tube 624 may include an opening 659 through the wall 655 of the tube 624. The flexible arm defining the keeper 646 includes a distal end 670 extending from the tube wall 655 at an angle and a free proximal end 672 that extends in or through the window 659 and into the lumen of the distal tube 624. Similar to FIG. 18, optionally, the distal tube 624 may include a cap or cover to cover the window 659. The cap may be attached in any suitable manner, such as adhesive, snap fit, etc.

Also similar to the catheter shown in FIGS. 14 and 15, the inner diameter of the proximal interlocking end portion 634 of the distal tube 624 may be smaller than the outer diameter of the proximal end portion 658 of the body 656 of the first interlocking member 652. For example, the proximal terminal end 661 of the distal tube 624 may be flared inwardly.

The extendable catheter 620 is stored and packaged in the collapsed configuration. After the user opens the package, the user moves the proximal and distal tubes 622, 624 away from each other, moving the proximal tube 622 out of the lumen of the distal tube 624. The diameter of latch's flange 664 is larger than the distance between the proximal end portion 672 of the arm defining the keeper 646 and the opposing inner surface 674 of the distal tube 624. As the user continues to move the proximal and distal tubes 622, 624 apart, the flange defining the latch 664 of the first interlocking member 652 contacts the flexible arm defining the keeper 646 of the second interlocking member 654. As the catheter 620 is pulled with sufficient force to move the flange defining the latch 664 past the flexible arm, the flange deforms and/or the arm flexes. When the flange has moved passed the proximal end 672 of the arm, the arm and/or the flange move back towards their original configurations. The distal surface 682 of the flange defining the latch 664 engages the proximal end 672 of the arm to lock the catheter 620 in the extended configuration. Furthermore, the proximal end 658 of the body 656 of the first interlocking member 652 contacts the proximal end 661 of the distal tube 624, which acts as a stop or stop mechanism to prevent the tubes from separating. The distance between the latch and the stop are such that there is minimal relative movement between the two tubes. The user then preforms catheterization with the catheter by inserting the catheter into the urethra. During catheterization, the distal tube 624 may have an insertable portion that may be inserted into the urethra.

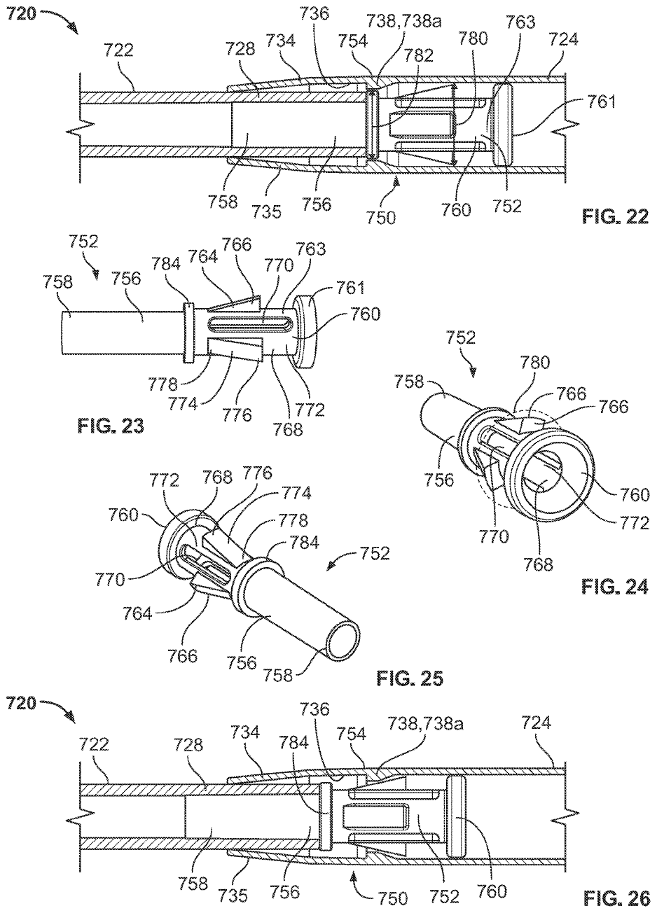

FIGS. 22 and 26-30 illustrate an extendable catheter 720 having an alternative interlocking mechanism 750. Similar to FIGS. 1 and 2, catheter 720 has a proximal tube 722 and a distal tube 724 wherein a portion of the proximal tube 722 is coaxial and resides within the distal tube 724. FIG. 22 shows the catheter 720 in the collapsed configuration and the interlocking mechanism 750 in an initial or unlocked state. FIG. 30 shows the catheter 720 in the extended configuration and the interlocking mechanism 750 in the locked state. In the locked state, the interlocking mechanism 750 has sufficient strength to prevent the catheter 720 from returning to the collapsed configuration during use. The interlocking mechanism 750 also prevents the proximal tube 722 and distal tube 724 from separating.

The interlocking mechanism 750 includes a first interlocking member 752 and a second interlocking member 754, which engage each other to lock the catheter 720 in an extended configuration or position. The first interlocking member 752 is associated with the distal interlocking end portion 728 of the proximal tube 722. In the illustrated embodiment, the first interlocking member 752 has a body 756 that includes a proximal end portion 758 and a distal end portion 760. The body 756 may be generally tubular with a longitudinal axis “X” (FIG. 30) and a lumen 762 (FIGS. 28 and 30) therethrough. The proximal end portion 758 of the first interlocking member 752 is connected to the distal end portion 728 of proximal tube 722. In one alternative, the distal end portion 728 of the proximal tube 122 is over-molded over the proximal end portion 758 of the first interlocking member 752. In another option, the proximal end portion 758 of the first interlocking member 752 is embedded in the distal end portion 728 of the proximal tube 722. In yet another alternative, the distal end portion 728 of the proximal tube 722 is friction or heat fit over the proximal end portion 758 of the first interlocking member 752.

Referring to FIGS. 23-25, the first interlocking member 752 includes one or more latches 764 extending radially outward from the wall 768 of the body 756. In the illustrated embodiment, the latches comprise fins 766 which may have a generally triangular cross-section and/or have the shape of a triangular prism. The wall 768 at the distal end portion 760 of the first interlocking member 752 is flexible and elastic. In the illustrated embodiment, the wall 768 of the distal end portion 760 includes a plurality of longitudinal openings 770, such as cavities, slots, or cut-outs, which aid in allowing the wall 768 to flex. The flexibility and elasticity of the wall may be varied by varying the material of the wall, the thickness of the wall and the size and shape of the opening. The structures of the wall 768 between the openings 770 define columns 772 which are flexible and elastic. The columns 772 may be compressible inward and then return to their initial state. For example, when force is applied to the columns, they may be compressed inward toward the longitudinal axis.

The fins 766 extend longitudinally along the columns 772 and radially outwardly from the columns 772. In the illustrated embodiment, the fins 766 have an outer surface 774 that is ramped or angled with respect the surface of the wall 768, the columns 772 and/or the longitudinal axis “X” of the body 758. The outer surface 774 angles radially inwardly toward the surface the wall 768 from distal end 776 of the fin to the proximal end 778 of the fin (FIGS. 23 and 25).

Referring back to FIGS. 22 and 26-30, the second interlocking member 754 may be defined by or at the proximal end portion 734 of the distal tube 724. The inner surface 736 of the proximal end portion 734 may include a keeper 738. The keeper 738 comprises one or more projections 738a extending into the lumen of the distal tube 724. The projection(s) 738a may be integral with the wall of the distal tube 724. In one alternative, the projection 738a may be a continuous, circumferential projection. In another alternative, the projection(s) 738a may be a plurality of projections. The projection(s) 738a may be integral with the distal tube 724. Alternatively, the projection(s) 738a may an insert or a component of an insert that is located within the proximal end of the distal tube 724.

The maximum combined outer radial boundary 780 defined by fins 766 is larger than the maximum combined inner radial boundary 782 defined by the projection(s) 738a of the keeper 738 (FIGS. 22 and 24). Furthermore, the inner surface 736 and outer surface 735 of the proximal end portion 734 of the distal tube 724 may be tapered radially inwardly in the proximal direction. For example, the outer surface 735 of the proximal end portion 734 of the distal tube 724 may be tapered to form a smooth transition with the outer surface 737 of the proximal tube 722. Furthermore, the inner surface 736 of the proximal end portion 734 of the distal tube 724 may be tapered so as to have a diameter that is smaller than an outer diameter of a portion 784 of the body 756 of the first interlocking member 752. In one alternatively, the body includes a circumferential projection 784 extending radially from the body 756. The circumferential projection 784 has an outer diameter larger than at least a portion of the tapered proximal end 734 of the distal tube 724. Contact between the tapered distal end 734 and the circumferential projection 784 prevent the tubes 722, 724 from separating. In one alternative, distal tube 724 may be made from component parts wherein the tapered proximal end 734 and the projection(s) 738a may be components of an insert that is connected to the remaining section of the distal end tube 724. Furthermore, the terminal distal end 761 of the body 756 may have a larger cross-section than an adjacent section 763 of the distal end portion 760 (FIGS. 22 and 23). For example, the distal end portion 760 may include a large flange that is has a larger outer boundary than the adjacent section 763. The terminal distal end 761 contacts the inner surface of the distal tube 724. This may assist in stabilizing the body 756 within the distal tube 724 and serve as an additional safety stop that prevents the tubes 722, 724 from separating.

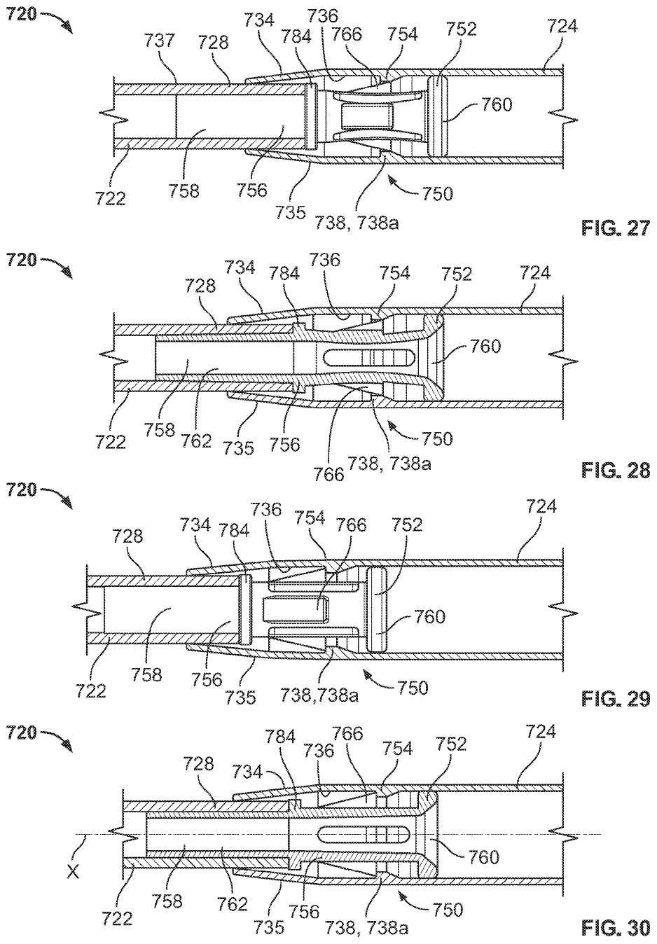

The extendable catheter 720 is stored and packaged (not shown) in the collapsed configuration shown in FIG. 22. After the user opens the package, the user moves the proximal and distal tubes 722, 724 away from each other, moving the proximal tube 722 out of the lumen of the distal tube 724. The maximin combined diameter of the fins 766 of the latch is larger than the combined inner diameter of the keeper's projection(s) 738a. As the user continues to move the proximal and distal tubes 722, 724 apart, the first interlocking member 752 contacts the projection(s) 738a of the keeper 738 of the second interlocking member 754. For example, the fins 766 of the first interlocking member 752 contact the projection(s) 738a of the second interlocking member 754. The ramped or angled outer surfaces 774 of the fins 766 engages the projection(s) 738a (FIG. 26). As the tubes 722, 724 continue to be moved apart and the ramped outer surfaces 774 of the fins 766 move along the projection(s) 738a, force is placed on the columns 772. The columns 772 flex or collapse inward allowing the fins 766 to pass by the projection(s) 738a (FIGS. 27 and 28). After the distal ends 776 of the fins 766 pass the projection(s) 738a, the columns 772 move back toward their initial state and the distal ends 776 of the fins 766 engage the projection(s) 738a to lock the catheter 720 in the extended configuration (FIGS. 29 and 30). Furthermore, the circumferential projection 784 of the body 756 of the first interlocking member 752 contacts the tapered inner surface 736 of the proximal end 734 of the distal tube 724, which acts as a stop or stop mechanism to prevent the tubes 722, 724 from separating. The distance between the latch and the stop is such that there is minimal relative movement between the two tubes. The user then preforms catheterization with the catheter by inserting the catheter into the urethra. During catheterization, the proximal end 734 of the distal tube 724 may have an insertable portion that may be inserted into the urethra.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

What is claimed is:

1. A urinary catheter that has a collapsed configuration and an extended configuration, comprising:
a proximal tube having a proximal insertion end portion and a distal interlocking end portion, the proximal tube including at least one drainage opening in the proximal insertion end portion and a lumen extending from the at least one drainage opening to the distal interlocking end portion;
a distal tube having a distal drainage end portion, a proximal interlocking end portion and a lumen extending from the proximal interlocking end portion to the distal drainage end portion;
wherein the proximal tube is displaceably arranged in the lumen of the distal tube such that the proximal tube and distal tube can be moved relative to each other from a collapsed configuration to an extended configuration;
an interlocking mechanism comprising a first interlocking member and a second interlocking member that engage to lock the proximal and distal tubes in the extended configuration;
the first interlocking member being associated with the distal interlocking end portion of the proximal tube, the first interlocking member including a body having a proximal end, a distal end and a generally tubular shape having a longitudinal axis, at least one radially extending latch extending from the body of the first interlocking member;
the second interlocking member being associated with the proximal interlocking end portion of the distal tube, the second interlocking member including at least one keeper; and
wherein when the proximal and distal tubes are moved into the extended configuration, the at least one radially extending latch of the first interlocking mechanism and the keeper of the second interlocking mechanism engage to lock the proximal and distal tubes in the extended configuration and prevent the proximal and distal tubes from moving to the collapsed configuration.

2. The urinary catheter of claim 1, wherein the first interlocking member includes a body having a flexible, elastic wall.

3. The urinary catheter of claim 2, wherein the wall includes openings and columns between the openings.

4. The urinary catheter of claim 2, wherein the first interlocking member further includes a plurality of fins extending radially from the flexible, elastic wall.

5. The urinary catheter of claim 3, further including fins extending from the columns.

6. The urinary catheter of claim 4, wherein the fins have a triangular prism shape.

7. The urinary catheter of claim 4, wherein the at least one keeper of the second interlocking member comprising one or more projections extending from an inner surface of the distal tube.

8. The urinary catheter of claim 7, wherein a maximum combined outer radial boundary defined by the fins is larger than a maximum combined inner radial boundary defined by the one or more projections.

9. The urinary catheter of claim 1, wherein the second interlocking member and the distal tube are separate and distinct components.

10. The urinary catheter of claim 9, wherein the second interlocking member comprises a generally tubular body having a proximal end, a distal end and a generally tubular having a longitudinal axis.

11. (canceled)

12. The urinary catheter of claim 10, wherein when in the proximal and distal tubes are in the extended configuration, the first interlocking member is located within the lumen of the second interlocking member.

13. The urinary catheter of claim 1, wherein the proximal end of the body of the first interlocking member has an outer surface and at least a portion of the at least one radially extending latch extends radially beyond the outer surface of the proximal end of the body of the first interlocking member.

14. The urinary catheter of claim 1, wherein the first interlocking member is a separate and the proximal tube are separate components.

15. (canceled)

16. The urinary catheter of claim 1, wherein a channel in an inner surface of the second interlocking member defines the keeper, and the latch engages the channel.

17. The urinary catheter of claim 1, wherein a shoulder extending inward from an inner surface of the second interlocking member defines the keeper.

18. The urinary catheter of claim 1, wherein the keeper of the second interlocking member comprises one or more arms having a proximal end extending at an angle.

19. The urinary catheter of claim 1, wherein the keeper of the second interlocking member comprises one or more arms extending at an angle from an inner surface of the distal tube, wherein a distal end of the one or more arms extends from the inner surface of the distal tube and a proximal end of the one or more arms extends at an angle into the lumen of the distal tube.

20. The urinary catheter of claim 19, wherein the one or more arms comprises a single arm.

21. (canceled)

22. (canceled)

23. The urinary catheter of claim 1, wherein the interlocking mechanism further includes a stop mechanism that prevents the proximal tube from completely separating.

24. (canceled)

25. (canceled)

26. (canceled)

27. (canceled)

28. The urinary catheter of claim 1, wherein the at least a portion of the distal tube is configured to be insertable into a urethra.

29. (canceled)
